(19) United States
(12) Patent Application Publication
(10) Pub. No.: US 2016/0120785 A1
(43) Pub. Date: May 5, 2016

(54) USE OF SPECIFIC ACRYLATES COPOLYMER AS SPF BOOSTER

(71) Applicant: L'OREAL, Paris (FR)

(72) Inventors: Susan HALPERN CHIRCH, Basking Ridge, NJ (US); Kevin NIKITCZUK, Skillman, NJ (US); Anil SHAH, East Windsor, NJ (US)

(21) Appl. No.: 14/531,207

(22) Filed: Nov. 3, 2014

(51) Int. Cl. A61K 8/81; A61K 8/37; A61K 8/35; A61Q 17/04; A61K 8/40

(52) U.S. Cl. CPC A61K 8/8152; A61Q 17/04; A61K 8/40; A61K 8/35; A61K 8/37; A61K 2800/592

(57) ABSTRACT

A sunscreen composition containing: (a) a polyacrylate booster comprising at least one acrylates copolymer having a weight average molecular weight ranging from about 75,000 to 140,000 g/mol and a Tg ranging from −20 to 50° C.; and (b) a UV filter system containing a combination of UV filters comprised of: octocrylene:avobenzone:oxybenzone:octisalate:homosalate; wherein the ratio of each filter relative to avobenzone is as follows:
the ratio of octocrylene to avobenzone is 1.6:1.0 to 2.4:1.0,
the ratio of oxybenzone to avobenzone 1.0:1.0 to 1.6:1.0,
the ratio of octisalate to avobenzone is 0.8:1.0 to 1.3:1.0, and
the ratio of homosalate to avobenzone is 2.8:1.0 to 4.3:1.

### FIELD OF THE INVENTION

The present invention relates to the use of a booster capable of synergistically boosting the SPF of a sunscreen composition. More particularly, the invention relates to the use of a booster comprised of an acrylates copolymer having a weight average molecular weight of from about 75,000 to 140,000 g/mol, and a Tg of from about −20 to 50° C. which has shown to provide a synergistic boost in SPF when combined with a novel UV filter system.

### BACKGROUND OF THE INVENTION

The negative effects of exposure to ultraviolet (“UV”) light are well-known. Prolonged exposure to sunlight causes damage such as sunburn to the skin and dries out hair making it brittle. When skin is exposed to UV light having a wavelength of from about 290 nm to about 400 nm, long term damage can lead to serious conditions such as skin cancer.

UV light also contributes to aging by causing free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products. The end result is a loss of skin elasticity and the appearance of wrinkles. This process is commonly referred to as photo-aging.

Sunscreens can be used to protect against UV damage and delay the signs of aging. The degree of UV protection afforded by a sunscreen composition is directly related to the amount and type of UV filters contained therein. The higher the amount of UV filters, the greater the degree of UV protection. Nevertheless, it is desirable to achieve the best photo protection efficacy with the lowest amount of UV filters. The inventors of the instant disclosure discovered ways to attain SPFs that were not previously attainable with such low amounts of overall UV filters.

### BRIEF SUMMARY OF THE INVENTION

The present disclosure relates to sunscreen compositions that have low amounts of UV filters yet excellent Sun Protection Factors (SPF). Typically, the more UV filters included in a sunscreen composition the higher the SPF. The inventors discovered that the association of a specific type of polyacrylate booster, together with a specific type of UV filter system, yields a synergistic interaction which provides an unexpectedly surprising boost in SPF values.

The UV filter system of the present disclosure comprises the following combination of UV filters: octocrylene, avobenzone, oxybenzone, octisalate, and homosalate; wherein the ratio of each filter relative to avobenzone is as follows:

the ratio of octocrylene to avobenzone is 1.6:1.0 to 2.4:1.0;

the ratio of oxybenzone to avobenzone 1.0:1.0 to 1.6:1.0;

the ratio of octisalate to avobenzone is 0.8:1.0 to 1.3:1.0; and

the ratio of homosalate to avobenzone is 2.8:1.0 to 4.3:1.

In particular, the ratio of each filter relative to avobenzone is about: 2.0:1.0:1.3:1.1:3.6 (octocrylene:avobenzone:oxybenzone:octisalate:homosalate).

In one embodiment the UV filters are present in the following percentages by weight relative to the entire weight of the sunscreen composition:

2 to 7 wt. % octocrylene;

1 to 3 wt. % avobenzone;

0 to 6 wt. % oxybenzone;

1 to 5 wt. % octisalate; and

2 to 10 wt. % homosalate.

In another embodiment the UV filters are present in the following percentages by weight relative to the entire weight of the sunscreen composition: about 5.6 wt. % octocrylene; about 2.8 wt. % avobenzone; about 3.6 wt. % oxybenzone; about 3.0 wt. % octisalate; and about 10.0 wt. % homosalate.

The polyacrylate booster of the present disclosure encompasses an acrylates copolymer having a weight average molecular weight ranging from about 75,000 to 140,000 g/mol and a Tg ranging from about −20 to 50° C.

The present disclosure is also directed to methods of making a sunscreen composition containing minimal amounts of UV filters while providing sufficient SPF values and corresponding protection from ultraviolet radiation.

### DETAILED DESCRIPTION OF THE INVENTION

Other than in the operating examples and unless otherwise stated, all numbers expressing quantities of ingredients and/or reaction conditions are to be understood as being modified in all instances by the term “about”.

Where the following terms are used in this specification, they are used as defined below.

The terms “comprising,” “having,” and “including” are used in their open, non-limiting sense.

The terms “a” and “the” are understood to encompass the plural as well as the singular.

As used herein, the expression “at least one” means one or more and thus includes individual components as well as mixtures/combinations.

“Cosmetically acceptable” means that the item in question is compatible with any keratinous substrate. For example, “cosmetically acceptable carrier” means a carrier that is compatible with any keratinous substrate.

A “physiologically acceptable medium” means a medium which is not toxic and can be applied to the skin, lips, hair, scalp, lashes, brows, nails or any other cutaneous region of the body. The composition of the instant disclosure may especially constitute a cosmetic or dermatological composition.

The phrase “essentially free” refers to less than or equal to 0.5, 0.1, 0.05 or 0.01 wt. %.

The phrase “stable emulsion” refers to a composition that does not undergo phase separation up to a temperature of 45° C. for at least two weeks.

### Polyacrylate Booster

In one embodiment, the polyacrylate booster is an acrylates copolymer having a weight average molecular weight ranging from about 75,000 to 140,000 g/mol, preferably ranging from about 84,000 to 125,000 g/mol, and most preferably ranging from about 88,000 to 120,000 g/mol, and a Tg ranging from about −20 to 50° C., preferably from about −10 to 40° C., and most preferably from about 0 to 20° C.

A particularly preferred acrylates copolymer for use in the present invention is one having a weight average molecular weight of from about 93,000 to 114,000 g/mol, and a Tg of about 13.6° C., sold under the tradename EPITEX™ 66 Polymer by Dow Chemical in the form of an aqueous polyacrylate emulsion.

The polyacrylate film former of the present invention is typically employed in an amount ranging from about 0.1% to about 10% by weight, preferably from about 0.25% to about 5% by weight, and most preferably from about 0.5% to about 2% by weight, based on the total weight of the composition.

### UV Filter System

The UV filter system of the present invention is comprised of the following combination of UV filters: octocrylene, avobenzone, oxybenzone, octisalate, and homosalate; wherein the ratio of each filter relative to avobenzone is as follows:

the ratio of octocrylene to avobenzone is 1.6:1.0 to 2.4:1.0;

the ratio of oxybenzone to avobenzone 1.0:1.0 to 1.6:1.0;

the ratio of octisalate to avobenzone is 0.8:1.0 to 1.3:1.0; and

the ratio of homosalate to avobenzone is 2.8:1.0 to 4.3:1.

In particular, the ratio of each filter relative to avobenzone is about: 2.0:1.0:1.3:1.1:3.6 (octocrylene:avobenzone:oxybenzone:octisalate:homosalate).

In one embodiment the UV filters are present in the following percentages by weight relative to the entire weight of the sunscreen composition:

from 1, 2, 3, 4, or 5 to 6, 7, 8, 9, or 10 wt. % octocrylene;

from 1, 2, or 3 to 3, 4, or 5 wt. % avobenzone;

from 0, 1, 2, or 3 to 3, 4, 5, or 6 wt. % oxybenzone;

from 1, 2, 3, or 4 to 5 wt. % octisalate; and

from 1, 2, 4, 6, 8, or 10 to 10, 12, 14.15 wt. % homosalate.

In another embodiment the UV filters are present in the following percentages by weight relative to the entire weight of the sunscreen composition: about 5.6 wt. % octocrylene; about 2.8 wt. % avobenzone; about 3.6 wt. % oxybenzone; about 3.0 wt. % octisalate; and about 10 wt. % homosalate.

It has been surprisingly discovered that the above-disclosed specific type of polyacrylate film former, when combined with the above-disclosed UV filter system, yields a synergistic boost in SPF of the composition.

The present disclosure is also directed to methods for protecting a keratinous substrate from ultraviolet radiation and to methods of absorbing ultraviolet light. Such methods encompass applying the sunscreen composition of the present invention onto a keratinous substrate and subjecting the keratinous substrate to ultraviolet radiation.

The present disclosure is also directed to a method of making a sunscreen composition involving combining the above-disclosed specific-type of polyacrylate film former with the above-disclosed UV filter system.

According to one embodiment, the sunscreen composition of the present invention contains from about 1% to about 40% by weight, preferably from about 5% to about 30% by weight, and most preferably from about 7% to about 25% by weight, of a UV filter system.

### Oils/Emollients

Examples of oils/emollients that may be included in the sunscreen compositions include: hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, coriander oil, castor oil, avocado oil, caprylic/capric acid triglycerides, for instance those sold by the company Stearineries Dubois or those sold under the names Miglyol® 810, 812 and 818 by the company Dynamit Nobel, jojoba oil, shea butter oil and caprylyl glycol; synthetic esters and ethers, especially of fatty acids, for instance purcellin oil, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name Eldew® SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam® oil, or the mixture of n-undecane (C₁₁) and of n-tridecane (C₁₃) sold under the reference Cetiol® UT by the company Cognis; fluoro oils that are partially hydrocarbon-based and/or silicone-based, for instance those described in document JP-A-2 295 912; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes; mixtures thereof.

Additional examples include benzoic acid esters of C₉-C₁₅ alcohols, isononyl iso-nonanoate, C₁₂-C₁₅ alkyl benzoate, or any combinations thereof.

Specific examples of oils/emollients include cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C₉-C₁₅ alcohols, isononyl iso-nonanoate, alkanes, mineral oil, silicone, dimethyl polysiloxane, ether, polyoxypropylene butyl ether, polyoxypropylene cetyl ether, C₁₂-C₁₅ alkyl benzoate, aryl alkyl benzoate, Isopropyl Lauroyl sarcosinate, and any combinations thereof.

Examples of hydrophilic organic solvents that may be included in the sunscreen compositions include:
monohydric C1-08 alcohols such as ethanol, propanol, butanol, isopropanol, isobutanol;
polyethylene glycols from 6 to 80 ethylene oxides such as propylene glycol, isoprene glycol, butylene glycol, glycerol, sorbitol;
mono or di-alkyl isosorbides such as dimethyl isosorbide;

Examples of amphiphilic organic solvents include: polypropylene glycol (PPG) like propylene glycol alkyl ester or alkyl ether of PPG like PPG-23 oleyl ether and PPG-36 oleate.

The above lists are only examples and not limiting.

The total amount of oils/emollient present in the compositions is typically about 0.1, 0.5, 1.0, or 2.5 wt. %, to about 5.0, 7.5, 10.0, 15.0, 20.0, or 30 wt. % of the total weight of the composition.

### Emulsifiers

The sunscreen compositions typically include at least one emulsifier such as an amphoteric, anionic, cationic or nonionic emulsifier, used alone or as a mixture, and optionally a co-emulsifier. The emulsifiers are chosen in an appropriate manner according to the emulsion to be obtained (W/O or O/W). The emulsifier and the co-emulsifier are generally present in the composition in a proportion ranging from 0.3% to 30% by weight and preferably from 0.5% to 20% by weight relative to the total weight of the composition.

For W/O emulsions, examples of emulsifiers that may be mentioned include dimethicone copolyols, such as the mixture of cyclomethicone and dimethicone copolyol sold under the trade name DC 5225 C by the company Dow Corning, and alkyl dimethicone copolyols such as the lauryl dimethicone copolyol sold under the name Dow Corning 5200 Formulation Aid by the company Dow Corning, and the cetyl dimethicone copolyol sold under the name Abil® EM 90 by the company Goldschmidt. A cross-linked elastomeric solid organopolysiloxane comprising at least one oxyalkylene group, such as those obtained according to the procedure of Examples 3, 4 and 8 of U.S. Pat. No. 5,412,004 and of the examples of U.S. Pat. No. 5,811,487, especially the product of Example 3 (synthesis example) of U.S. Pat. No. 5,412,004, such as the product sold under the reference KSG 21 by the company Shin-Etsu, may also be used as surfactants for W/O emulsions.

For O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof.

The fatty acid esters of a sugar that can be used as nonionic amphiphilic lipids can be chosen in particular from the group comprising esters or mixtures of esters of a C₈-C₂₂ fatty acid and of sucrose, of maltose, of glucose or of fructose, and esters or mixtures of esters of a C₁₄-C₂₂ fatty acid and of methylglucose.

The C₈-C₂₂ or C₁₄-C₂₂ fatty acids forming the fatty unit of the esters that can be used in the emulsion comprise a saturated or unsaturated linear alkyl chain having, respectively, from 8 to 22 or from 14 to 22 carbon atoms. The fatty unit of the esters can be chosen in particular from stearates, behenates, arachidonates, palmitates, myristates, laurates, caprates and mixtures thereof.

By way of example of esters or of mixtures of esters of a fatty acid and of sucrose, of maltose, of glucose or of fructose, mention may be made of sucrose monostearte, sucrose distearate, sucrose tristearate and mixtures thereof, such as the products sold by the company Croda under the name Crodesta™ F50, F70, F110 and F160 having, respectively, an HLB (Hydrophilic Lipophilic Balance) of 5, 7, 11 and 16; and, by way of example of esters or of mixtures of esters of a fatty acid and of methylglucose, mention may be made of the disearate of methylglucose and of polyglycerol-3, sold by the company Goldschmidt under the name Tego-care® 450. Mention may also be made of glucose monoesters or maltose monoesters, such as methyl O-hexadecanoyl-6-D-glucoside and O-hexadecanoyl-6-D-maltoside.

The fatty alcohol ethers of a sugar that can be used as nonionic amphiphilic lipids can be chosen in particular form the group comprising ethers or mixtures of ethers of a C₈-C₂₂ fatty alcohol and of glucose, of maltose, of sucrose or of fructose, and ethers or mixtures of ethers of a C₁₄-C₂₂ fatty alcohol and of methylglucose. They are in particular alkylpolyglucosides.

The C₈-C₂₂ or C₁₄-C₂₂ fatty alcohols forming the fatty unit of the ethers that can be used in the emulsion of the instant disclosure comprise a saturated or unsaturated linear alkyl chain having, respectively, from 8 to 22 or from 14 to 22 carbon atoms. The fatty unit of the ethers can be chosen in particular from decyl, cetyl, behenyl, arachidyl, stearyl, palmityl, myristyl, lauryl, capryl and hexadecanoyl units, and mixtures thereof such as cetearyl.

By way of example of fatty alcohol ethers of a sugar, mention may be made of alkylpolyglucosides, such as decylglucoside and laurylglucoside sold, for example, by the company Henkel under the respective names Plantaren® 2000 and Plantaren® 1200, cetostearylglucoside, optionally as a mixture with cetostearyl alcohol, sold, for example, under the name Montanov™ 68 by the company Seppic, under the name Tego-care® CG90 by the company Goldschmidt and under the name Emulgade KE3302 by the company Henkel, and also arachidylglucoside, for example in the form of the mixture of arachidyl and behenyl alcohols and of arachidylglucoside sold under the name Montanov™ 202 by the company Seppic.

Use is more particularly made, as nonionic amphiphilic lipid of this type, of sucrose monostearate, sucrose distearate, sucrose tristearate and mixtures thereof, the distearate of methylglucose and of polyglycerol-3, and alkylpolyglucosides.

The glycerol fatty esters that can be used as nonionic amphiphilic lipids can be chosen in particular from the group comprising the esters formed from at least one acid comprising a saturated linear alkyl chain having from 16 to 22 carbon atoms, and from 1 to 10 glycerol units. Use may be made of one or more of these glycerol fatty esters in the emulsion of the instant disclosure.

These esters may be chosen in particular from stearates, behenates, arachidates, palmitates and mixtures thereof. Stearates and palmitates are preferably used.

By way of example of a surfactant that can be used in the emulsion of the instant disclosure, mention may be made of decaglycerol monostearate, distearate, tristearate and pentastearate (10 glycerol units) (CTFA names: polyglyceryl-10 stearate, polyglyceryl-10 distearate, polyglyceryl-10 tristearate, polyglyceryl-10 pentastearate), such as the products sold under the respective names Nikkol Decaglyn 1-S, 2-S, 3-S and 5-S by the company Nikko, and diglyceryl monostearate (CTFA name: polyglyceryl-2 stearate) such as the product sold by the company Nikko under the name Nikkol DGMS.

The sorbitan fatty esters that can be used as nonionic amphiphilic lipids chosen in particular from the group comprising esters of a C₁₆-C₂₂ fatty acid and of sorbitan and oxyethylenated esters of a C₁₆-C₂₂ fatty acid and of sorbitan. They are formed from at least one fatty acid comprising at least one saturated linear alkyl chain, having, respectively, from 16 to 22 carbon atoms, and from sorbitol or from ethoxylated sorbitol. The oxyethylenated esters generally comprise from 1 to 100 ethylene oxide units, and preferably from 2 to 40 ethylene oxide (EO) units.

These esters can be chosen in particular from stearates, behenates, arachidates, palmitates and mixtures thereof. Stearates and palmitates are preferably used.

By way of example of sorbitan fatty ester and of an oxyethylenated sorbitan fatty ester, mention may be made of sorbitan monostearate (CTFA name: sorbitan stearate) sold by the company ICI under the name Span® 60, sorbitan monopalmitate (CTFA name: sorbitan palmitate) sold by the company ICI under the name Span® 40, or sorbitan 20 EO tristearate (CTFA name: polysorbate 65) sold by the company ICI under the name Tween® 65.

The ethoxylated fatty ethers are typically ethers made up of 1 to 100 ethylene oxide units and of at least one fatty alcohol chain having from 16 to 22 carbon atoms. The fatty chain of the ethers can be chosen in particular from behenyl, arachidyl, stearyl and cetyl units, and mixtures thereof, such as cetearyl. By way of example of ethoxylated fatty ethers, mention may be made of ethers of behenyl alcohol comprising 5, 10, 20 and 30 ethylene oxide units (CTFA names: beheneth-5, beheneth-10, beheneth-20 and beheneth-30), such as the products sold under the names Nikkol BBS, BB10, BB20 and BB30 by the company Nikko, and the ether of stearyl alcohol comprising 2 ethylene oxide units (CTFA name: steareth-2), such as the product sold under the name Brij™ 72 by the company ICI.

The ethoxylated fatty esters that can be used as nonionic amphiphilic lipids are esters made up of 1 to 100 ethylene oxide units and of at least one fatty acid chain comprising from 16 to 22 carbon atoms. The fatty chain of the esters can be chosen in particular from stearate, behenate, arachidate and palmitate units, and mixtures thereof. By way of example of ethoxylated fatty esters, mention may be made of the ester of stearic acid comprising 40 ethylene oxide units, such as the product sold under the name Myrj™ 52 (CTFA name: PEG-40 stearate) by the company ICI, and the ester of behenic acid comprising 8 ethylene oxide units (CTFA name: PEG-8 behenate), such as the product sold under the name Compritol® HD5 ATO by the company Gattefosse.

The block copolymers of ethylene oxide and of propylene oxide that can be used as nonionic amphiphilic can be chosen in particular from poloxamers and in particular from Poloxamer 231, such as the product sold by the company ICI under the name Pluronic® L81 of formula (V) with x=z=6, y=39 (HLB 2); Poloxamer 282, such as the product sold by the company ICI under the name Pluronic® L92 of formula (V) with x=z=10, y=47 (HLB 6); and Poloxamer 124, such as the product sold by the company ICI under the name Pluronic® L44 of formula (V) with x=z=11, y=21 (HLB 16).

As nonionic amphiphilic lipids, mention may also be made of the mixtures of nonionic surfactants described in document EP-A-705593, incorporated herein for reference.

Suitable hydrophobically-modified emulsifiers include, for example, inulin lauryl carbamate, commercially available from Beneo Orafti under the tradename Inutec® SP1.

The above lists are only examples and not limiting.

The total amount of emulsifier present in the compositions is typically in an amount of about 0.1, 0.2, or 0.5 wt. % to about 4.0, 5.0, 6.0, or 7.5 wt. %, based on the total weight of the composition.

### Gelling Agent

Gelling agents may also be included in the sunscreen compositions. Examples of suitable hydrophilic gelling agents include carboxyvinyl polymers such as the Carbopol® products (carbomers) and the Pemulen™ products (acrylate/C10-C30-alkylacrylate copolymer); polyacrylamides, for instance the cross-linked copolymers sold under the names Sepigel™ 305 (CTFA name: polyacrylamide/C13-14 isoparaffin/Laureth 7) or Simulgel™ 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, which are optionally cross-linked and/or neutralized, for instance the poly(2-acrylamido-2-methylpropanesulfonic acid) (CTFA name: ammonium polyacryldimethyltauramide); cellulose-based derivatives such as hydroxyethyl-cellulose; polysaccharides and especially gums such as xanthan gum; and mixtures thereof.

Lipophilic gelling agents (thickeners) that may be mentioned include modified clays such as hectorite and its derivatives, for instance the products sold under the name bentone.

In some instances, the gelling agent is ammonium acryloyldimethyltaurate/steareth-25 methacrylate crosspolymer, commercially available from Clariant under the tradename Aristoflex® HMS.

The above lists are only examples and not limiting.

The gelling agent is typically used in an amount of about 0.02% to about 1.5% by weight, from about 0.05% to about 1.0% by weight, or about 0.1 to about 0.5% by weight, based on the total weight of the composition.

### Additional Sunscreen Filters (Protective Agents)

The sunscreen compositions can include additional sunscreen filters such as, for example, mineral UV filters. Examples of mineral UV filters include pigments and nanopigments (mean size of the primary particles is generally is from 5 nm to 100 nm or from 10 nm to 50 nm) of treated or untreated metal oxides such as, for example, nanopigments of titanium oxide (amorphous or crystallized in rutile and/or anatase form), of iron oxide, of zinc oxide, of zirconium oxide or of cerium oxide. The treated nanopigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal (titanium or aluminum) alkoxides, polyethylene, silicones, proteins (collagen or elastin), alkanolamines, silicon oxides, metal oxides, sodium hexametaphosphate, alumina or glycerol. The treated nanopigments may more particularly be titanium oxides treated with:
silica and alumina, such as the products “Microtitanium Dioxide MT 500 SA” and “Microtitanium Dioxide MT 100 SA” from the company Tayca, and the products “Tioveil™ Fin”, “Tioveil™ OP”, “Tioveil™ MOTG” and “Tioveil™ IPM” from the company Tioxide;
alumina and aluminum stearate, such as the product “Microtitanium Dioxide MT 100 T” from the company Tayca;
alumina and aluminum laurate, such as the product “Microtitanium Dioxide MT 100 S” from the company Tayca;
iron oxides and iron stearate, such as the product “Microtitanium Dioxide MT 100 F” from the company Tayca;
silica, alumina and silicone, such as the products “Microtitanium Dioxide MT 100 SAS”, “Microtitanium Dioxide MT 600 SAS” and “Microtitanium Dioxide MT 500 SAS” from the company Tayca;
sodium hexametaphosphate, such as the product “Microtitanium Dioxide MT 150 W” from the company Tayca;
octyltrimethoxysilane, such as the product “T-805” from the company Degussa;
alumina and stearic acid, such as the product “UVT-M160” from the company Kemira;
alumina and glycerol, such as the product “UVT-M212” from the company Kemira;
alumina and silicone, such as the product “UVT-M262” from the company Kemira.

Other titanium oxide nanopigments treated with a silicone are TiO₂ treated with octyltrimethylsilane and for which the mean size of the elementary particles is between 25 and 40 nm, such as the product sold under the trade name “T805” by the company Degussa Silices, TiO₂ treated with a polydimethylsiloxane and for which the mean size of the elementary particles is 21 nm, such as the product sold under the trade name “70250 Cardre UF TiO2SI3” by the company Cardre, anatase/rutile TiO₂ treated with a polydimethylhydrogenosiloxane and for which the mean size of the elementary particles is 25 nm, such as the product sold under the trade name “Microtitanium Dioxide USP Grade Hydrophobic” by the company Color Techniques.

Uncoated titanium oxide nanopigments are sold, for example, by the company Tayca under the trade names “Microtitanium Dioxide MT 500 B” or “Microtitanium Dioxide MT 600 B”, by the company Degussa under the name “P 25”, by the company Wackher under the name “Oxyde de titane transparent PW”, by the company Myoshi Kasei under the name “UFTR”, by the company Tomen under the name “ITS” and by the company Tioxide under the name “Tioveil™ AQ”.

The uncoated zinc oxide nanopigments are, for example:
those sold under the name “Z-Cote®” by the company Sunsmart;
those sold under the name “Nanox®” by the company Elementis; and
those sold under the name “Nanoguard WCD 2025” by the company Nanophase Technologies.

The coated zinc oxide nanopigments are, for example:
those sold under the name “Zinc Oxide CS-5” by the company Toshibi (ZnO coated with polymethylhydrogenosiloxane);
those sold under the name “Nanoguard Zinc Oxide FN” by the company Nanophase Technologies (as a 40% dispersion in Finsolv® TN, C₁₂-C₁₅ alkyl benzoate);
those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name “NFD Ultrafine ZNO” by the company Daikin (ZnO coated with perfluoroalkyl phosphate and copolymer based on perfluoroalkylethyl as a dispersion in cyclopentasiloxane);
those sold under the name “SPD-Z1” by the company Shin-Etsu (ZnO coated with silicone-grafted acrylic polymer, dispersed in cyclodimethylsiloxane); those sold under the name “Escalol™ Z100” by the company ISP (alumina-treated ZnO dispersed in an ethylhexyl methoxycinnamate/PVP-hexadecene/methicone copolymer mixture);
those sold under the name “Fuji ZNO-SMS-10” by the company Fuji Pigment (ZnO coated with silica and polymethylsilsesquioxane); and
those sold under the name “Nanox® Gel TN” by the company Elementis (ZnO dispersed at a concentration of 55% in C₁₂-C₁₅ alkyl benzoate with hydroxystearic acid polycondensate).

The uncoated cerium oxide nanopigments are sold under the name “Colloidal Cerium Oxide” by the company Rhone-Poulenc. The uncoated iron oxide nanopigments are sold, for example, by the company Arnaud under the names “Nanoguard WCD 2002 (FE 45B)”, “Nanoguard Iron FE 45 BL AQ”, “Nanoguard FE 45R AQ” and “Nanoguard WCD 2006 (FE 45R)” or by the company Mitsubishi under the name “TY-220”. The coated iron oxide nanopigments are sold, for example, by the company Arnaud under the names “Nanoguard WCD 2008 (FE 45B FN)”, “Nanoguard WCD 2009 (FE 45B 556)”, “Nanoguard FE 45 BL 345” and “Nanoguard FE 45 BL” or by the company BASF under the name “Transparent Iron Oxide”.

Mixtures of metal oxides may also be used, especially of titanium dioxide and of cerium dioxide, including the silica-coated equal-weight mixture of titanium dioxide and of cerium dioxide, sold by the company Ikeda under the name “Sunveil A”, and also the alumina, silica and silicone-coated mixture of titanium dioxide and of zinc dioxide, such as the product “M 261” sold by the company Kemira, or the alumina, silica and glycerol-coated mixture of titanium dioxide and of zinc dioxide, such as the product “M 211” sold by the company Kemira.

The above lists are only examples and not limiting.

The compositions according to the instant disclosure may be prepared according to techniques that are well known to those skilled in the art, in particular those intended for the preparation of emulsions of oil-in-water or water-in-oil type. They may be in particular in the form of a simple or complex emulsion (O/W, W/O, O/W/O or W/O/W emulsion) such as a cream or milk, in the form of a gel or a cream-gel, or in the form of a lotion.

The instant disclosure will be better understood from the examples that follow, all of which are intended for illustrative purposes only and are not meant to limit the scope of the instant disclosure in any way.

### Examples

TABLE 1
Inventive Example

| Phase | US INCI name | Example 1 |
|---|---|---|
| A | Water | Q.S. |
| | EDTA | 0.100 |
| | Propylene Glycol | 2.500 |
| B | Avobenzone | 2.800 |
| | Octocrylene | 5.600 |
| | Octisalate | 3.000 |
| | Oxybenzone | 3.600 |
| | Homosalate | 10.000 |
| | Ethylhexyl Palmitate | 5.000 |
| | Isohexadecane | 7.500 |
| | Glyceryl Stearate (and) PEG-100 Stearate | 1.000 |
| | (ARLACEL ™ 165) | |
| | Dimethicone | 1.700 |
| | Carbomer (ASHLAND ™ 980) | 0.200 |
| | Phenoxyethanol (and) Methylparaben (and) | 0.800 |
| | Ethylparaben (and) Propylparaben (and) | |
| | Isobutylparaben (and) Butylparaben (PHENONIP) | |
| C | Water | 0.500 |
| | Triethylamine | 0.350 |
| D | Acrylates Copolymer (EPITEX ™ 66) | 2.060 |
| | TOTAL | 100% |

| | |
|---|---|
| SPF (in-vitro) | 21.6 |

TABLE 2
Inventive Example without Polyacryate Booster

| Phase | US INCI name | Example 2 |
|---|---|---|
| A | Water | Q.S. |
| | EDTA | 0.100 |
| | Propylene Glycol | 2.500 |
| B | Avobenzone | 2.800 |
| | Octocrylene | 5.600 |
| | Octisalate | 3.000 |
| | Oxybenzone | 3.600 |
| | Homosalate | 10.000 |
| | Ethylhexyl Palmitate | 5.000 |
| | Isohexadecane | 7.500 |
| | Glyceryl Stearate (and) PEG-100 Stearate | 1.000 |
| | (ARLACEL ™ 165) | |
| | Dimethicone | 1.700 |
| | Carbomer (ASHLAND ™ 980) | 0.200 |
| | Phenoxyethanol (and) Methylparaben (and) | 0.800 |
| | Ethylparaben (and) Propylparaben (and) | |
| | Isobutylparaben (and) Butylparaben (PHENONIP) | |
| C | Water | 0.500 |
| | Triethylamine | 0.350 |
| | TOTAL | 100% |

| | |
|---|---|
| SPF (in-vitro) | 10.32 |

TABLE 3
Comparative Examples

| Phase | US INCI name | Example 3 | Example 4 |
|---|---|---|---|
| A | Water | Q.S. | Q.S. |
| | EDTA | 0.100 | 0.100 |
| | Propylene Glycol | 2.500 | 2.500 |
| B | Octylmethoxycinnamate | 7.500 | 7.500 |
| | Octocrylene | 10.000 | 10.000 |
| | Octisalate | 4.000 | 4.000 |
| | Oxybenzone | 4.000 | 4.000 |
| | Ethylhexyl Palmitate | 5.000 | 5.000 |
| | Isohexadecane | 7.500 | 7.500 |
| | Glyceryl Stearate (and) PEG-100 Stearate | 1.000 | 1.000 |
| | (ARLACEL ™ 165) | | |
| | Dimethicone | 1.700 | 1.700 |
| | Carbomer (ASHLAND ™ 980) | 0.200 | 0.200 |
| | Phenoxyethanol (and) Methylparaben | 0.800 | 0.800 |
| | (and) Ethylparaben (and) Propylparaben | | |
| | (and) Isobutylparaben (and) Butylparaben | | |
| | (PHENONIP) | | |
| C | Water | 0.500 | 0.500 |
| | Triethylamine | 0.350 | 0.350 |
| D | Acrylates Copolymer (EPITEX ™ 66) | 0.000 | 2.060 |
| | TOTAL | 100% | 100% |

| | | |
|---|---|---|
| SPF (in-vitro) | 18.49 | 18.79 |

The examples in Tables 1, 2, and 3 were prepared according to the procedure outlined below.

1) Heat Phase A to 80° C.

2) While heating add Phase D premix into Phase A.

3) Heat Phase B to 80° C.

4) Add phase B into Phase A. Homogenized for 20 minutes.

5) Begin cooling to room temperature.

6) While cooling add phase C at 50° C. Homogenize for 5 minutes.

7) Add phase E at 30-35° C. and mix.

8) Add phase F at 30° C. and mix.

9) Stop mixing at 25° C.

In vitro SPF measurements were performed according to the following procedure: each sample was applied to a PMMA plate (polymethyl methacrylate plate) with a draw down bar to control the thickness and the homogeneity of the film; the in vitro SPF was measured using a Labsphere 2000. Each measurement was made 6 times (6 times on each plate) on 3 plates for each composition.

TABLE 4
SPF Boost in Inventive Composition

| | | SPF | SPF |
|---|---|---|---|
| Sample | Description | (in vitro) | Boost |
| Example 2 | Composition without acrylates | 10.32 | — |
| | copolymer | | |
| Example 1 | Composition with acrylates copolymer | 21.6 | +109% |
| | (EPITEX ™ 66) | | |

TABLE 5
SPF Change in Comparative Composition

| | | SPF | SPF |
|---|---|---|---|
| Sample | Description | (in vitro) | Change |
| Example 3 | Composition without acrylates | 18.49 | — |
| | copolymer | | |
| Example 4 | Composition with acrylates copolymer | 18.79 | no |
| | (EPITEX ™ 66) | | change |

What is claimed is:

1. A sunscreen composition comprising: (a) a polyacrylate booster comprising at least one acrylates copolymer having a weight average molecular weight ranging from about 75,000 to 140,000 g/mol and a Tg ranging from −20 to 50° C.; and (b) a UV filter system containing a combination of UV filters comprised of: octocrylene:avobenzone:oxybenzone:octisalate:homosalate; wherein the ratio of each filter relative to avobenzone is as follows:
the ratio of octocrylene to avobenzone is 1.6:1.0 to 2.4:1.0,
the ratio of oxybenzone to avobenzone 1.0:1.0 to 1.6:1.0,
the ratio of octisalate to avobenzone is 0.8:1.0 to 1.3:1.0, and
the ratio of homosalate to avobenzone is 2.8:1.0 to 4.3:1.

2. The sunscreen composition according to claim 1, wherein the ratio of each filter relative to avobenzone is about: 2.0:1.0:1.3:1.1:3.6 (octocrylene:avobenzone:oxybenzone:octisalate:homosalate).

3. The sunscreen composition according to claim 1 having an SPF of at least 15.

4. The sunscreen composition according to claim 1 having an SPF of at least 30.

5. The sunscreen composition according to claim 1 having an SPF of at least 50.

6. The sunscreen composition according to claim 1 having an SPF of at least 75.

7. The sunscreen composition according to claim 1 having an SPF of at least 100.

8. The sunscreen composition according to claim 1 having an SPF of at least 125.

9. The sunscreen composition according to claim 1 having an SPF of at least 150.

10. The sunscreen composition according to claim 1 comprising:
about 5.6 wt. % octocrylene;
about 2.8 wt. % avobenzone;
about 3.6 wt. % oxybenzone;
about 3 wt. % octisalate; and
about 10 wt. % homosalate.

11. The sunscreen composition according to claim 1 wherein (a) is an acrylates copolymer having weight average molecular weight ranging from about 84,000 to 125,000 g/mol.

12. The sunscreen composition according to claim 1 wherein (a) is an acrylates copolymer having a weight average molecular weight ranging from about 88,000 to 120,000 g/mol.

13. The sunscreen composition according to claim 1 wherein (a) is an acrylates copolymer having a Tg ranging from about −10 to 40° C.

14. The sunscreen composition according to claim 1 wherein (a) is an acrylates copolymer having a Tg ranging from about 0 to 20° C.

15. The sunscreen composition according to claim 1 wherein (a) is an acrylates copolymer having a weight average molecular weight of from about 93,000 to 114,000 g/mol, and a Tg of about 13.6° C.

16. The sunscreen composition according to claim 1 wherein (a) is present in the composition in an amount of from about 0.1% to about 10% by weight, based on the total weight of the composition.

17. The sunscreen composition according to claim 1 wherein (a) is present in the composition in an amount of from about 0.25% to about 5% by weight, based on the total weight of the composition.

18. The sunscreen composition according to claim 1 wherein (a) is present in the composition in an amount of from about 0.5% to about 2% by weight, based on the total weight of the composition.

19. A sunscreen composition comprising: (a) from about 0.5% to about 2% by weight, based on the total weight of the composition, of a polyacrylate booster comprising at least one acrylates copolymer having a weight average molecular weight ranging from about 93,000 to 114,000 g/mol and a Tg of about 13.6° C.; and (b) a UV filter system containing a combination of UV filters comprised of: octocrylene:avobenzone:oxybenzone:octisalate:homosalate; wherein the ratio of each filter relative to avobenzone is about: 2.0:1.0:1.3:1.1:3.6 (octocrylene:avobenzone:oxybenzone:octisalate:homosalate).

20. A method of protecting a keratinous substrate from ultraviolet radiation comprising applying a sunscreen composition according to claim 1 to the keratinous substrate.

21. A method of making a sunscreen composition comprising: (a) providing a polyacrylate booster comprising at least one acrylates copolymer having a weight average molecular weight ranging from about 75,000 to 140,000 g/mol and a Tg ranging from −20 to 50° C.; (b) providing a UV filter system containing a combination of UV filters comprised of: octocrylene avobenzone oxybenzone:octisalate:homosalate; wherein the ratio of each filter relative to avobenzone is as follows:
the ratio of octocrylene to avobenzone is 1.6:1.0 to 2.4:1.0,
the ratio of oxybenzone to avobenzone 1.0:1.0 to 1.6:1.0,
the ratio of octisalate to avobenzone is 0.8:1.0 to 1.3:1.0, and
the ratio of homosalate to avobenzone is 2.8:1.0 to 4.3:1
and (c) combining (a) and (b) to make the sunscreen composition.

22. The method according to claim 21, wherein the ratio of each filter relative to avobenzone is about: 2.0:1.0:1.3:1.1:3.6 (octocrylene:avobenzone:oxybenzone:octisalate:homosalate).

23. The method according to claim 21 having an SPF of at least 15.

24. The method according to claim 21 having an SPF of at least 30.

25. The method according to claim 21 having an SPF of at least 50.

26. The method according to claim 21 having an SPF of at least 75.

27. The method according to claim 21 having an SPF of at least 100.

28. The method according to claim 21 having an SPF of at least 125.

29. The method according to claim 21 having an SPF of at least 150.

30. The method according to claim 21 comprising:
about 5.6 wt. % octocrylene;
about 2.8 wt. % avobenzone;
about 3.6 wt. % oxybenzone;
about 3 wt. % octisalate; and
about 10 wt. % homosalate.

31. The method according to claim 21 wherein (a) is an acrylates copolymer having weight average molecular weight ranging from about 84,000 to 125,000 g/mol.

32. The method according to claim 21 wherein (a) is an acrylates copolymer having a weight average molecular weight ranging from about 88,000 to 120,000 g/mol.

33. The method according to claim 21 wherein (a) is an acrylates copolymer having a Tg ranging from about −10 to 40° C.

34. The method according to claim 21 wherein (a) is an acrylates copolymer having a Tg ranging from about 0 to 20° C.

35. The method according to claim 21 wherein (a) is an acrylates copolymer having a weight average molecular weight of from about 93,000 to 114,000 g/mol, and a Tg of about 13.6° C.

36. The method according to claim 21 wherein (a) is present in the composition in an amount of from about 0.1% to about 10% by weight, based on the total weight of the composition.

37. The method according to claim 21 wherein (a) is present in the composition in an amount of from about 0.25% to about 5% by weight, based on the total weight of the composition.

38. The method according to claim 21 wherein (a) is present in the composition in an amount of from about 0.5% to about 2% by weight, based on the total weight of the composition.

39. A method of making a sunscreen composition comprising: (a) providing from about 0.5% to about 2% by weight, based on the total weight of the composition, of a polyacrylate booster comprising at least one acrylates copolymer having a weight average molecular weight ranging from about 93,000 to 114,000 g/mol and a Tg of about 13.6° C.; (b) providing a UV filter system containing a combination of UV filters comprised of: octocrylene:avobenzone:oxybenzone:octisalate:homosalate; wherein the ratio of each filter relative to avobenzone is about: 2.0:1.0:1.3:1.1:3.6 (octocrylene:avobenzone:oxybenzone:octisalate:homosalate); and (c) combining (a) and (b) to form the sunscreen composition.
